(19) United States
(12) Patent Application Publication
(10) Pub. No.: US 2021/0322325 A1
(43) Pub. Date: Oct. 21, 2021

(54) CONTROLLED DRUG RELEASE FORMULATION

(71) Applicant: Fachhochschule Nordwestschweiz, Muttenz (CH)

(72) Inventors: Georgios IMANIDIS, Bottmingen (CH); Michael LANZ, Wabern (CH); Georg LIPPS, Riehen (CH); Valeria PAREDES, Riehen (CH)

(21) Appl. No.: 17/272,512

(22) PCT Filed: Sep. 3, 2019
(86) PCT No.: PCT/EP2019/073462

§ 371 Date: Mar. 1, 2021

(30) Foreign Application Priority Data
Sep. 6, 2018 (EP) 18192852.4
Dec. 13, 2018 (EP) 18212186.3

(51) Int. Cl. A61K 9/50; A61K 31/513; A61K 31/4745; A61K 31/282; A61K 31/436; A61K 38/13; A61K 31/196; A61K 31/58; A61K 31/7068; A61K 39/395; A61K 45/06; A61K 47/12; A61K 47/36; A61K 9/20; A61K 9/00

(52) U.S. Cl. CPC A61K 9/5036; A61K 9/0053; A61K 31/513; A61K 31/4745; A61K 31/282; A61K 31/436; A61K 38/13; A61K 31/196; A61K 31/58; A61K 31/7068; A61K 39/3955; A61K 45/06; A61K 9/5089; A61K 47/12; A61K 47/36; A61K 9/2081; A61K 9/5026

(57) ABSTRACT

Pharmaceutical formulation dosage form (1) with a core (2) encapsulated by at least one shell (3) and comprising at least one active pharmaceutical ingredient (4), wherein the at least one active pharmaceutical ingredient (4) is embedded in said core (2) of the pharmaceutical formulation dosage form (1), preferably in that said core (2) is formed by a matrix based on xyloglucan (5) containing said active pharmaceutical ingredient (4), and wherein said shell (3) is a pH-responsive coating.

### TECHNICAL FIELD

The present invention relates to pharmaceutical formulation dosage forms, in particular for the oral administration of active pharmaceutical ingredients to be delivered selectively to the colon as well as methods for making such pharmaceutical formulation dosage forms and dosage regimens suitable for the corresponding dosage forms.

### PRIOR ART

WO-A-2015158771 discloses compositions comprising synergic combinations of xyloglucans and plant or animal proteins, which are useful in the treatment of intestinal disorders. Tablets for the treatment of diarrhea are proposed based on xyloglucan, pea protein or gelatin.

US-A-2017088557 describes a process for the preparation of rifaximin T, an antibiotic used to treat traveler's diarrhea, irritable bowel syndrome, and hepatic encephalopathy, a pharmaceutical composition comprising said rifaximin form as well as typical formulation ingredients such as microcrystalline cellulose, HPMC, glyceryl stearate, sodium starch glycolate, and its use for treating inflammations and infections.

WO-A-2007122374 discloses a delayed release coating comprising a mixture of a first material selected from starch; amylose; amylopectin; chitosan; chondroitin sulfate; cyclodextrin; dextran; pullulan; carrageenan; scleroglucan; chitin; curdulan and levan, and a second material which has a pH threshold at about pH 5 or above, is used to target release of a drug from a core to the intestine, particularly the colon.

US-A-2018000740 discloses pharmaceutical particulates which release a pharmaceutical compound into the colon following oral administration. A particulate comprises a core comprising a pharmaceutical compound, an inner coating surrounding the core, wherein the inner coating comprises a pharmaceutically acceptable polysaccharide that is susceptible to enzymatic digestion by one or more enzymes present colonic microflora, and an outer coating surrounding the inner coating, wherein the outer coating comprises a polymer which is stable at upper gastrointestinal pH but can dissolve at pH>6. The core of a particulate can further comprise an excipient such as a diluent, a binder, a disintegrant, a lubricant, a glidant or a combination thereof. Particulates can comprise pharmaceutical compounds for treating colonic diseases such as C. difficile infection, ulcerative colitis, colon cancer, and Crohn's disease.

Paulraj et al in “Bioinspired capsules based on nano-cellulose, xyloglucan and pectin—The influence of capsule wall composition on permeability properties” (Acta Biomaterialia 69 (2018) 196-205) present a study in which hollow microcapsules are built up in a layer by layer process using cellulose nano-fibrils and xyloglucan-amyloid or cellulose nano-fibrils, xyloglucan-amyloid and apple pectin as material for the individual layers. It is found that the corresponding wall structure is selectively permeable, depending on the electrolyte concentration, to a system such as dextrane and use of the corresponding microcapsules for pharmaceutical purposes are envisaged as future applications.

Mishra et al. (Int J Pharm Sci, 3(1), 139-142) describe the use of tamarind seed polysaccharide as tablet matrix and studied the release of ibuprofen in in-vitro test. Ibuprofen release is accelerated in the presence of rat caecal content.

Svagan et al in “Rhamnogalacturonan-I Based Microcapsules for Targeted Drug Release” (PLOS ONE, Dec. 19, 2016) disclose a method for making microcapsules based on Rhamnogalacturonan-I cross-linked by a diisocyanate and provide evidence that the corresponding microcapsules can uptake model systems as well as evidence that the microcapsules are released under corresponding enzymatic conditions.

Yoo et al. (Arch Pharm Res Vol 28, 6, p 736-742) describe the use of a degalactosylated xyloglucan for the sustained release of indomethacin. By treatment of xyloglucan with a beta-galactosidase the terminal galactose residues are removed leading to a change of the rheological and colloidal properties of the polymer. Degalactosylated xyloglucan exhibits thermally reversible sol-gel transitions a property not observed with unmodified xyloglucan (Brun-Graeppi, Amanda K. Andriola Silva et al. 2010. “Study on the Sol-Gel Transition of Xyloglucan Hydrogels.” Carbohydrate Polymers 80(2): 555-62.). The degalactosylated xyloglucan was mixed with indomethacin and allowed to form hydrogels. The hydrogel beads were dried and coated with Eudragit L100. The obtained formulation mainly released indomethacin in the small intestine as shown by in-vitro experiments simulating the gastric passage.

WO-A-2012038898 discloses gastro-resistant tablets containing rifaximin, obtained by means of gastro-resistant micro-granules characterized in that they inhibit the rifaximin release at pH values between 1.5 and 4.0, and they allow its release at pH values between 5.0 and 7.5, the processes for their obtainment and their use in the treatment and the prevention of diseases directly or indirectly deriving from inflammatory bowel diseases. The active pharmaceutical ingredient is embedded in a matrix of various constituents, including silica, methacrylic acid methyl methacrylate, talc, titanium dioxide, iron oxide, microcrystalline cellulose, magnesium stearate, et cetera. The tablets may be provided with a film coating based on hydroxy propyl methylcellulose and titanium dioxide.

### SUMMARY OF THE INVENTION

It is therefore an object of the present invention to provide and propose a new pharmaceutical formulation dosage form for (per)oral administration and allowing for a targeted release of an active pharmaceutical ingredient (API), in particular in the colon, including situations with the aim of immunomodulation or immunosuppression or of establishing, re-establishing and/or modifying the balance of the microbiome population in the colon or the physiology of the lower gastrointestinal tract.

The proposed pharmaceutical formulation dosage form is a core-shell type tablet which comprises a core encapsulated by at least one shell and at least one active pharmaceutical ingredient, wherein the at least one active pharmaceutical ingredient is embedded in said core of the pharmaceutical formulation dosage form.

According to the present invention, at least one of said core and said shell is, at least partly, based on xyloglucan. Preferably said core is formed by a matrix based on xyloglucan, or essentially consisting of xyloglucan, containing said active pharmaceutical ingredient. Xyloglucan is thus acting as an excipient and/or additive in solid pharmaceutical dosage forms for oral/per-oral administration, including tablets, e.g. compressed tablets or molded tablets, which can be un-coated, film-coated or sugar coated, to control and target delivery of active pharmaceutical ingredients for local therapeutic action in the gastrointestinal tract including the colon.

Furthermore, said shell is a pH-responsive coating, and preferably the xyloglucan, if only in the shell, should be in the layer which is forming the pH-responsive coating or should be in a coating layer which is inside of the pH-responsive coating.

The invention thus entails the use of xyloglucan as a matrix forming material for the manufacture of solid dosage forms such as tablets in which active pharmaceutical ingredient (API) is physically embedded. When in contact with intestinal fluid, the xyloglucan matrix or coating does not disintegrate instead slowly forming a highly viscous gel-like solution or gluey mass that impedes the release of API. Upon arrival of the dosage form in the colon, xyloglucan is digested by the microflora triggering release of the API. Hence, API or drug delivery specifically to the colon is achieved eliciting efficient API targeting. Xyloglucan is a polysaccharide of plant cell wall origin. A xyloglucan quality purified from Tamarindus indica seeds is used although material of other plant sources may also apply. Xyloglucan was shown to be digested by several Bacteroides species which is the most abundant genus in the gut microbiome.

To minimize contact of xyloglucan with intestinal fluid before it reaches the colon and further lessen premature release of API, the dosage form is coated with a pH-responsive film that dissolves at pH of at least 6.8.

The combination of xyloglucan used as embedding matrix material for API with pH sensitive film coating creates a redundancy of release controlling mechanisms that is intended to optimize the therapeutic index of the API. The coating is designed to dissolve at a slightly acidic to neutral pH that occurs under all circumstances in the small intestine to absolutely assure that the film is removed before the dosage form reaches the colon. After the coating is dissolved, release of API that would otherwise take place prematurely in the small intestine is impeded by the property of xyloglucan to not disintegrate forming instead a highly viscous mass. Only digestion of xyloglucan by the colonic microbiota triggers release of the API providing highly efficient active ingredient or drug targeting.

Controlled release in the gastrointestinal tract relying only on pH-sensitive coatings provides highly variable results. This is due to the intra and inter-individual variability of pH in the intestine, the dependence of pH on the intake of food, etc. Thus, an early dissolution of the coating before the arrival of the dosage form in the large intestine results in systemic absorption of the active ingredient and therefore its loss for colon specific delivery and local therapeutic effect, and the generation of systemic side effects, i.e., a worsened therapeutic index. A failure of the coating to dissolve in the small intestine on the other hand results in elimination of the intact dosage form in the faeces.

Use of materials forming a matrix as a means to prevent release of active ingredient or drug substance in the small intestine requires effective barrier formation by these materials in the aqueous environment of the small intestine and their efficient degradation by the microbiome of the colon.

Prior art has used enteric coating alone and xyloglucan alone to prevent release in the stomach and to enhance release in the intestine but failed to demonstrate the combination of the dual use of enteric coating and xyloglucan is useful for colonic delivery. The proposed dual release mechanism also specifically addresses the intra- and inter-individual variability of the gut conditions for which no solutions have yet been proposed.

Efficient active ingredient or drug targeting to the colon by per-oral administration with minimal active ingredient or drug release in the small intestine and therefore minimal absorption into the systemic circulation and maximal delivery of API in the colon for local therapeutic effect is thus achieved. This is required and advantageous for therapeutic treatment of inflammatory bowel disease, colon cancer, Clostridium difficile infection and further conditions of the large intestine that benefit from local rather than systemic active ingredient or drug application, but also for immunomodulation or immunosuppression or for the purpose of establishing, re-establishing and/or modifying the balance of the microbiome population in the colon or the physiology of the lower gastrointestinal tract.

New pharmaceutical products are thus made available for specific colonic active ingredient or drug delivery by per-oral administration. Therapeutic areas include inflammatory bowel disease and colon cancer, but also immunomodulation or immunosuppression. Existing active pharmaceutical ingredients (API) for these indications may primarily be used, although utilization of new chemical entities is also possible.

The expression active pharmaceutical ingredient (API) in the context of the present application includes conventional pharmaceutical compounds, be it small molecules or large molecules such as for example antibody-based pharmaceuticals, in particular for treatmentasa tabletss or for immunomodulation or immunosuppression.

However active pharmaceutical ingredient in the present context also includes any kind of material for the purpose of establishing, re-establishing and/or modifying the balance of the microbiome population in the colon. These include:
1) selected strains of bacteria including spores and/or mixture of strains;
2) nutrients for colonic microorganisms like fermentation substrates, nitrogen source, trace elements (Fe) etc.;
3) modulators of bacterial growth including vitamins, hormones, antibiotics, toxins etc.;
4) compounds which influence the composition of the microbiome by favoring and/or disfavoring the growth, viability or colonization of selected strains.

So the term API also generally includes compounds which have a beneficial effect on the physiology of the lower gastrointestinal tract.

Current delivery modalities do not achieve the level of colon targeting that is required for best active ingredient or drug therapeutic index comprising maximal therapeutic effect or immunomodulation or immunosuppression or re-establishing and/or modifying the balance of the microbiome population, and minimized side effects.

Depending upon the porosity of the tablet, as can be seen further below, after a short burst the API (5-ASA) is released with zero order kinetics afterwards. The same release kinetics is observed regardless whether the tablets are coated or not. Uncoated tablets release 5-ASA immediately; coated tablets release as soon as the enteric coatings is dissolved (depending upon pH and specific coating type and thickness used).

Tablets with enteric coating use the coating to protect the tablet and to prevent disintegration and API release during the gastric passage. Commercial 5-ASA tablets mostly have a poly-(acrylate-methacrylate) coating. The coatings differ in composition and the release is specifically triggered at a certain time point depending on the pH which initiates the dissolution of the coating. Thus, the time point of release is critically dependent upon pH. In other words, the delay intended by the tablets is largely influenced by the pH in the intestine, which in turn is influenced by a number of physiological factors. Consequently, a reliable delayed release with such a formulation is not attainable especially in patients suffering Morbus Crohn and IBD.

In contrast, the tablets proposed here are formulated in such a way to prevent release under weakly acidic to almost neutral conditions as long as possible. This in principle bears the risk that the tablets do not disintegrate in the colon and are excreted more or less intact. However, the inclusion of xyloglucan as tablet matrix destabilizes the tablet core in the colon due to the action of the microbiome which specifically digests plant cell wall material. The purpose of the enteric coating for our technology is therefore to allow a stabilization of the tablet also along the small intestine. During the gastric and the small intestine passage the matrix core is wetted which could speed up the digestion of the xyloglucan in the colon by the resident colonic microbiome.

The combination of enteric coating and xyloglucan goes beyond a simple additive effect. Surprisingly, there is a synergetic effect on API (e.g. 5-ASA) release. The coated and the uncoated tablets release 3 to 4% of the API (e.g. 5-ASA) load per hour in the absence of the microbial enzyme (FIG. 4 (uncoated), FIG. 3, trace 0 U/mL and FIG. 5, trace 8 and 9 (coated)). In contrast, the uncoated tablets release under the same experimental conditions but in the presence of the microbial enzyme 7.5% of API (5-ASA) per hour and the coated tablet 15% per hour (FIG. 3, trace 1 U/mL). This more rapid release observed in the coated tablets allows for a more efficient colonic delivery.

The pharmaceutical formulation dosage form, typically a tablet, is preferably adapted for oral administration and for targeted release of the active pharmaceutical ingredient in the colon. To this end, preferably said shell is a pH-responsive coating dissolving only at a pH of more than 6.5, preferably of at least 6.7, more preferably of at least 6.8.

According to a first preferred embodiment, said at least one active pharmaceutical ingredient is embedded in said core of the pharmaceutical formulation dosage form in that said core is formed by a matrix based on xyloglucan containing said active pharmaceutical ingredient. So the shell may be free from xyloglucan, the API then being embedded in the core in a matrix based on or essentially consisting of xyloglucan.

Said shell may comprise alternatively or additionally at least one outer layer in the form of a pH responsive coating with at least one layer based on xyloglucan. If the shell comprises a layer based on xyloglucan, typically this is instead of having xyloglucan as the matrix component of the core. The core is then preferably formed by the API alone or the core contains the API in a matrix without xyloglucan. However it is also possible to have a shell layer based on xyloglucan as well as a core matrix based on xyloglucan.

In case of a shell layer based on xyloglucan said shell layer based on xyloglucan or further shell layers then include further components to provide for the pH-responsivity. In particular for the case where a pH-responsive outer coating of the shell is not based on xyloglucan, e.g. for the case where there is no xyloglucan forming the matrix of the API in the core, there can be at least one further inner shell layer based on xyloglucan.

The pharmaceutical formulation dosage form can be adapted for oral administration and for targeted release of the active pharmaceutical ingredient in the colon, and said shell may comprise at least one or consist of a pH-responsive coating dissolving only at a pH of more than 6.5, preferably of at least 6.7, more preferably of at least 6.8.

Said shell, in particular the at least one pH responsive coating thereof, can be based on synthetic polymers such as an anionic acrylate copolymer, preferably on an anionic copolymer based on methyl acrylate, methyl methacrylate and methacrylic acid, wherein preferably the ratio of the free carboxyl groups to the ester groups is in the range of 1:5-1:10, preferably in the range of 1:10, wherein preferably the anionic acrylate copolymer has a weight average molar mass (Mw) in the range of 200,000-400,000 g/mole, preferably in the range of 250,000-300,000 g/mole, one or a mixture of the following systems: biopolymers, in particular non watersoluble biopolymers, such as plant and/or animal derived biopolymers, including mixtures of free and esterified aliphatic and/or aromatic hydroxyacids,

Said shell, in particular the at least one pH responsive coating thereof, may consist of a mixture of an anionic acrylate copolymer, preferably on an anionic copolymer based on methyl acrylate, methyl methacrylate and methacrylic acid, wherein preferably the ratio of the free carboxyl groups to the ester groups is in the range of 1:5-1:10, preferably in the range of 1:10, wherein preferably the anionic acrylate copolymer has a weight average molar mass (Mw) in the range of 200,000-400,000 g/mole, preferably in the range of 250,000-300,000 g/mole, with further additives in a proportion of less than 25%, said further additives preferably being selected from the group consisting of polyoxyethylene and derivatives thereof, anionic surfactants, in particular sodium laurylsulfate, talc, dye, in particular iron(III)oxide, stabilizers, in particular triethyl citrate, The dry coating amount of the at least one pH responsive coating or of the whole shell can be in the range of 1-10, preferably in the range of 2.5-6 mg/cm2. In particular if the shell or at least one layer of the shell is based on xyloglucan the dry coating amount can also be much higher. For example, if the core is made of API alone, then the xyloglucan shell layer can be, by weight, up to as much as the core, e.g. in the range of 30-50% by weight of the core.

There can be provided only one single encapsulating pH responsive coating forming said shell.

Said matrix of the core may essentially or completely consist of xyloglucan, wherein preferably said xyloglucan is obtained from Tamarindus indica seeds and/or is cold water soluble and/or is amorphous.

The xyloglucan used as starting material can have a particle size (d50%) of at least 70 μm, preferably in the range of 70-150 μm, more preferably in the range of 80-110 μm. The xyloglucan can have a weight average molar mass (Mw) in the range of 400,000-500,000 g/mol.

The preferably said xyloglucan is fully cold water soluble, meaning it is fully soluble upon cold mixing of the starting materials at room temperature for a concentration of at least 1% w/v, preferably of at least 1.5 or 2% w/v in distilled water. Preferably this type of said xyloglucan is further fully amorphous and essentially free from impurities, in particular free from glucose and/or dextran, i.e. the purity of the starting material is at least 90% by weight, preferably a least 95% or at least 99%. If such a type of said xyloglucan is chosen the tablets do have a reduced tendency of disintegration and are thus more stable and provide for a more consistently controlled and reliable API release in the colon. In particular, tablets can be made which do not disintegrate even after 4 h or 6 h in water at room temperature (measured according to Ph. Eur.).

Surprisingly, the type of xyloglucan has a significant effect on its effect and suitability. We have worked with two highly purified but otherwise native xyloglucans Glyloid 2A (hot water soluble) and 3S (cold water soluble). The hot water soluble variety Glyloid 2A was processed for tablet core production. The tablet cores disintegrated rapidly. We have expected that the hot water soluble xyloglucan would be more suitable than the cold water soluble xyloglucan since the hot water soluble was expected to release the API less efficiently at room or body temperature. So one would expect that the hot-water soluble variety would be the preferred matrix as it would not dissolve at physiological pH and form a solid matrix impeding drug release while the cold-water-soluble variety would entail the considerable risk that the matrix would rapidly dissolve away in the intestine rendering a delayed or colonic delivery impossible. Surprisingly we found that tablets produced from hot-water soluble types disintegrated in water relatively fast (<1 hour) into small solid particles which due to the high surface then release the API rather quickly, while in contrast, the cold-water soluble xyloglucan tablets formed a viscous gluey mass in the perimeter that impeded drug release while the tablet remained intact for at least 24 hours.

Preferably the xyloglucan is therefore non-degalactosylated and/or native. Preferably the xyloglucan is a native, highly purified xyloglucan, more preferably of a cold-water soluble type.

The core may consist of
(A) 25-90%, preferably 40-90% by weight of xyloglucan;
(B) 10-60% by weight of at least one active pharmaceutical ingredient;
(C) 0-20% by weight, preferably 5-10% by weight of one or more pharmaceutically acceptable excipients selected from the group consisting of a diluent, a binder, an anti-adherent, a lubricant, a glidant and a combination thereof, wherein preferably the pharmaceutically acceptable excipient essentially consists of a binder or a binder and an anti-adherent, in particular the binder being selected as PVP and the anti-adherent as magnesium stearate.

The core may also consist of granules consisting of
(A) 25-90%, preferably 40-90% by weight of xyloglucan;
(B) 10-60% by weight of at least one active pharmaceutical ingredient;
(C) 0-20% by weight, preferably 5-10% by weight of one or more pharmaceutically acceptable excipients selected from the group consisting of a diluent, a binder, a lubricant, a glidant and a combination thereof, wherein preferably the pharmaceutically acceptable excipient essentially consists of a binder, in particular the binder being selected as PVP, which granules are compacted to form a core (before applying the shell, wherein preferably before compacting the granules are blended with an anti-adherent, preferably in the form of magnesium stearate.

Preferably the core is a single solid compressed core with a relative density of at least 0.7 (70%), preferably of at least 0.75 (75%) or at least 0.8 (80%).

The apparent density is determined based on the weight of the tablet (by weighing, room temperature, 23°, r.H. 65%) and the volume of the tablet calculated from the geometric form by geometric formulas.

The relative density (ρᵣ) or porosity is calculated by the following formula

$${ρ}_{r} = 1 - ɛ = \frac{{ρ}_{s c h}}{{ρ}_{solid}}$$

wherein the apparent density (ρ_{sch}) is determined from the weight and the volume (as above) and the solid density (ρ_{solid}) is measured by a gas pycnometer (model used here is a Multi-Pycnometer available from Quantachrome Instruments). The method with the gas pycnometer is e.g. described in the European Pharmacopoeia Ph. Eur. 8 (2.9.23, p 324).

Preferably the core and/or the whole pharmaceutical formulation dosage form has an average extension in the direction of the smallest diameter of at least 3 mm, preferably at least 4 mm, more preferably at least 4.5 or 5 mm. Preferably they have an average extension in the direction of the largest diameter of at least 8 mm, preferably of at least 10 mm, more preferably in the range of 10-14 mm or of 12 mm. The tablets are preferably compressed or moulded tablets and they can be of circular, oval or polygonal, in particular rectangular with rounded edges shape in the direction of the larger extension, and they can be flat faced plain, flat faced radius edged, flat faced bevel edged, standard convex face, compound cut. Inter alia for the mere size there is delay of release: Release from a spherical matrix with a radius of 1 and an active ingredient content of 570 (arbitrary units) shows a total release reached after 140 hours. A matrix half the size with radius 0.5 has an active ingredient content of 70 (due to the smaller volume, with identical density and other parameters) and releases the total amount of active ingredient in 40 hours. A matrix with a radius of 0.2 has an active ingredient content of 4.5 and shows a complete release in 6 hours. These are simulated (calculated) results based on the diffusion equation and demonstrate that normalized to the same total quantity of active ingredient, the larger (geometrically) the dosage form (tablets, beads, etc.), the slower the release process.

Preferably the core and/or the whole pharmaceutical formulation dosage form has a crushing force of at least 25 N, preferably of at least 40 N, more preferably of at least 100 N. The method for determining the crushing force of the tablets is also described in the European Pharmacopoeia Ph. Eur. 8 (2.9.8. p 299).

The weight ratio of the matrix of the core matrix based on xyloglucan to the at least one active pharmaceutical ingredient is preferably at least 1:2, preferably at least 1:1, more preferably at least 2:1.

The porosity of the core with xyloglucan as matrix can be in the range 10-35% (void volume percentage), and the degree of porosity can be used to control the release properties of the API.

The proposed pharmaceutical formulation dosage form can be used for the purpose of establishing, re-establishing and/or modifying the balance of the microbiome population in the colon or the physiology of the lower gastrointestinal tract, for immunomodulation or immunosuppression, or for the treatment of at least one of the following conditions: inflammatory bowel disease, in particular ulcerative colitis and/or Crohn's disease, Clostridium difficile infection, colon cancer, post colon surgical treatment.

The active pharmaceutical ingredient can be selected from the group consisting of: mesalazine, budesonide, capecitabine, fluorouracil, irinotecan, oxaliplatin, UFT, cetuximab, panitumumab. UFT is a dihydropyrimidine dehydrogenase inhibitory fluoropyrimidine drug, which combines uracil, a competitive inhibitor of DPD, with the 5-fluorouracil (5-FU) prodrug tegafur in a 4:1 molar ratio.

Further possible are immunomodulatory or immunosuppressive ingredients including immunosuppressive glucocorticoids, immunosuppressive cytostatics, immunosuppressive (poly- or monoclonal) antibodies, immunosuppressive drugs acting on immunophilins, interleukins, cytokines, chemokines, immunomodulatory imide drug. Possible are e.g. in particular tacrolimus, cyclosporine.

Also possible active pharmaceutical ingredients are materials for the purpose of establishing, re-establishing and/or modifying the balance of the microbiome population in the colon or compounds which have a beneficial effect on the physiology of the lower gastrointestinal tract, or combinations thereof.

The pharmaceutical formulation dosage form can be administered orally at least once a day, preferably twice a day, over a time span of at least one week, preferably at least two weeks, or at least two months or at least 1 year or even life-long.

Furthermore the present invention relates to a method for making a pharmaceutical formulation dosage form as given above, wherein in a first step xyloglucan, at least one active pharmaceutical ingredient, as well as if needed one or more pharmaceutically acceptable excipients are mixed and then compacted to form the core or mixed and treated to form granules, which are subsequently, if needed by first mixing the granules with a further treatment agent, compacted to form the core, wherein the mixing in both cases can take place using preferably a fluidised bed granulator or high shear mixer, and the core is subsequently coated in a second step with at least one coating forming a shell, wherein preferably the coating formulation is provided as a dispersion and is applied further preferably in a drum coater or using another method.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematic representation of the action mechanism;

FIG. 2 shows the release profile of different concentrations of the API in the matrix over time and conditions with mesalazine as API (5-ASA);

FIG. 3 shows the release profile of API in the presence of different concentrations of the xyloglucanase in the solution in the last phase over time and conditions with mesalazine as API (5-ASA);

FIG. 4 shows the property of xyloglucan to slow down active ingredient or drug release depending on the porosity of un-coated tablets;

FIG. 5 shows the release profile of API in the presence of different types of tablets with different thickness (amount) of the coating in solutions simulating the conditions of passage through the gastrointestinal tract over time with mesalazine as API (5-ASA).

### DESCRIPTION OF PREFERRED EMBODIMENTS

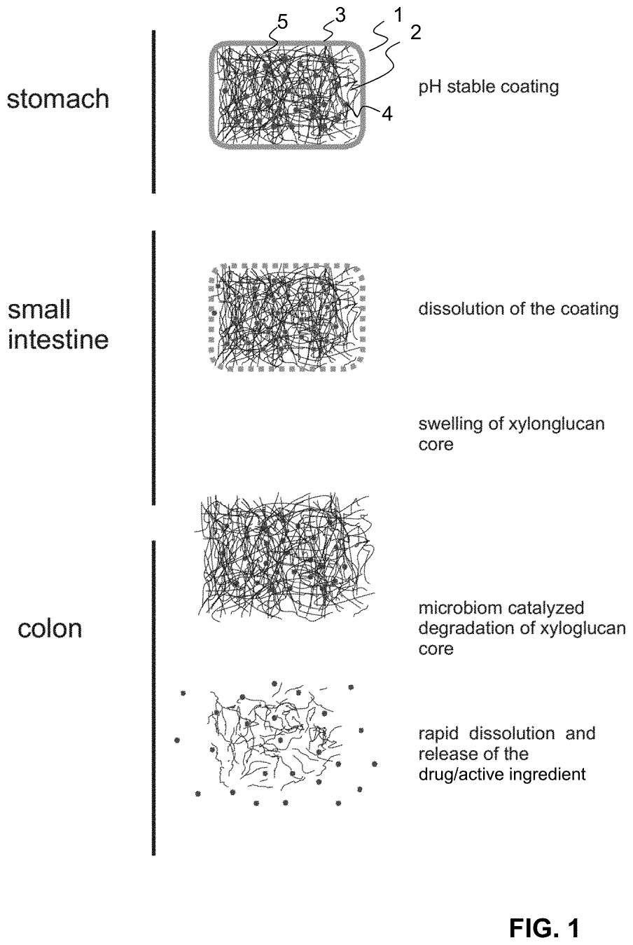

FIG. 1 schematically shows the working principle of the proposed pharmaceutical dosage form 1. The dosage form 1 comprises a core 2 which is encapsulated in a shell 3. The core comprises a matrix 5, in this particular case xyloglucan, in which the active pharmaceutical ingredient 4 (API) is embedded.

The materials of the core, in particular its matrix, as well as of the shell are adapted for selective release of the API in the colon. In this respect it is to be noted that in the stomach typically there is a pH of 1.2, and the average residence time in the stomach is about two hours. Then follows the proximal small intestine again with a typical residence time of two hours and an increased pH of 6.5. This is followed by the distal small intestine with again a typical average residence time of two hours and a pH of 6.8. Only then follows the colon, first with the ascending colon followed by the descending colon, the residence time here depends on various factors, the pH is still in the range of 6.8.

The shell 3 of the proposed formulation dosage form is adapted to only dissolve significantly once the pH increases above 6.5, typically reaches a value of at least 6.8. Correspondingly the core portion of the tablet only starts to dissolve in the small intestine. However that is not yet the place where the API is to be released. To this end that xyloglucan is forming the matrix of the core. Under the physiological conditions in the small intestine the core portion now essentially without coating swells and forms a highly viscous mass but does not release the active ingredient to a significant extent. Only once this swollen matrix still containing the API enters the colon with the different micro-organisms containing enzymes digesting xyloglucan the matrix is digested and disintegrates and then also the API is released in a targeted manner to the place where it shall develop its effects.

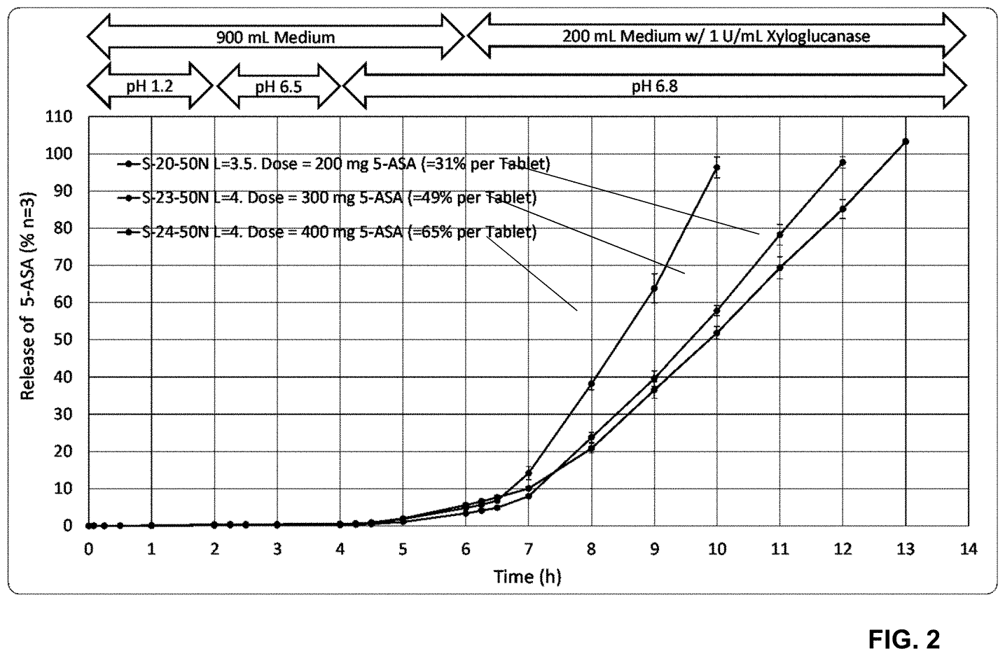

This is evidenced by the release profile illustrated in FIG. 2. In in vitro experiments (for the detail see further below) for the first two hours the corresponding tablet is subjected to a pH of 1.2 simulating stomach conditions. No release of the API can be detected. Subsequently for another two hours the conditions of the proximal small intestine are simulated with a pH of 6.5. Again no release of the API can be detected. Then for another two hours the conditions of the distal small intestine are simulated by increasing the pH value of 6.8 but still not changing the enzymatic surrounding. One can see that at this moment in time first small portion of the API is released, which is associated with the dissolution of the pH-dependent coating. After that time period, so after six hours counting from the start, also the enzymatic conditions are adapted to the ones as present in the colon, in particular xyloglucanase is introduced into the medium. As from this moment on, there is a sharp and targeted release of the API. As a matter of fact, the release is largely independent of the concentration of the API in the xyloglucan matrix.

### Material and Methods

### Tablet Production

A three step process was employed.

### 1. Granulation

Granulation was carried out either in a fluidized bed granulator or in a high shear mixer. The composition was as follows:

### Glyloid 3S (93-X) %

### 5-ASA (API) X %

### Polyvinylpyrrolidone (Kollidon 30) 7%

Three different compositions with the following values of X were used: 33.3%; 50%; 66.7%. For fluidized bed granulation with batch size of 600 g the first two ingredients were first blended in a Turbula mixer for 7 min. at 32 rpm and the third ingredient was dissolved in purified water in a concentration of 10% w/w. This solution was sprayed in the fluidized bed at a rate of 14 g/min at first that was reduced to 7.3 and then 9.7 g/min. The atomizing pressure was 1.3 to 1.5 bar. The air volume stream was at first 40 m3/h and was increased to 80 m3/h. The inlet temperature was 50° C. and the product temperature was approx. 25° C. throughout the liquid addition and increased to 29° C. during drying. The total process duration was approx. 65 min and the residual moisture was 6.8%. The granules were passed through a 1 mm mesh screen.

For high shear granulation with batch size of 300 g all three ingredients were first blended in a Turbula mixer for 7 min at 32 rpm. Purified water was sprayed in the mixer at a rate of 6.5 g/min and an atomizing pressure of 0.12 bar. The rotation rate of the main impeller was 220 rpm and of the chopper 2200 rpm. Between 110 and 170 g of water was added yielding an increase of the power consumption of the main impeller from 82 to 91-93 Watt. The granules were passed through a 1 mm mesh screen, dried in a tray drier at 50° C. to a residual moisture of <5% and passed again through a 0.85 mm mesh screen.

### 2. Tableting

Granulated compositions were blended with 0.5% Mg-stearate in a Turbula mixer for 2 min at 32 rpm. Tablets with a diameter of 12 mm, a radius of curvature of 9 mm and a diametric crushing force of 50 N were produced in a single punch eccentric tableting machine at a rate of 20 tablets per minute. The tablet weight was adjusted based on the API content of the compositions determined after granulation to between 600 and 630 mg to reach an API content of 200, 300 and 400 mg per tablet. The compression force of the upper punch was between 10 and 13 kN.

### 3. Coating

Tablets were coated with Eudragit FS-30-D in a drum coater with batch size of 600 g. The composition of the coating dispersion was as follows:

| | |
|---|---|
| Eudragit FS-30-D | 43% |
| Triethyl-citrate | 0.65% |
| Talc | 6.45% |
| Dye (iron III oxide) | 0.2% |
| Purified water | 49.7% |

The drum rotation speed was 20 rpm, the inlet air temperature 50° C., the product temperature 30-35° C., and the air volume stream 25-30 m3/h. The coating dispersion was sprayed at a rate of 4 g/min and an atomizing pressure of 1.3 bar. The nominal dry coating amount was L=5 mg/cm2 and the actual value was between L=3.5 and 4 mg/cm2.

### Material Characteristics

### Xyloglucan:

Brand name: Glyloid 3S and Glyloid 2A (DSP GOKYO FOOD & CHEMICAL Co., Ltd. Osaka, Japan)
Common name: Tamarind seed polysaccharide or tamarind seed gum
Chemical substance: Xyloglucan
Gras status declaration by FDA: GRN No. 503; Substance: Tamarind seed polysaccharide; Intended Use: Use as a thickener, stabilizer, emulsifier, and gelling agent in certain food categories; Notifier: DSP GOKYO FOOD & CHEMICAL Co., Ltd.; HERBIS OSAKA 20th Floor 2-5-25 Umeda Kita-ku, Osaka, 530-0001 Japan; Date of filing: Mar. 5, 2014 GRAS Notice (releasable information): 503; Date of closure: Aug. 12, 2014.

Physical-chemical properties declared by the supplier

| | Glyloid 2A | Glyloid 3S |
|---|---|---|
| Content | 80 - 99.99% | 90 - 99.99% |
| Glucose (inpurity) | 0.01 - 20% | 0.01 - 10% |
| Water solubility | Soluble | Soluble |
| Organic solvent | Not soluble | Not soluble |
| solubility | | |
| Molecular weight * | Approx. 470,000 | Approx. 470,000 |
| Physical form | White to light | White to light |
| | brown powder | brown powder |
| Loss on drying * | (more than of 3S quality) | 1.1% |
| Viscosity * | (less than of 3S quality) | 730 mPas |

* Values in the FDA document deviate

Physical-chemical properties determined on laboratory

| | Glyloid 2A | Glyloid 3S |
|---|---|---|
| Water solubility | Not completely | Soluble at room |
| | soluble at room | temperature |
| | temperature for | for 2% w/v |
| | 2% w/v | |
| | Soluble at 90° C. | |
| XRPD | Semi-amorphous | Amorphous |
| | (crystalline part | |
| | corresponds to | |
| | D-glucose) | |
| Particle size | d(10%) = 25 μm | d(10%) = 57.7 μm |
| | d(50%) = 60 μm | d(50%) = 91.4 μm |
| | d(90%) = 164 μm | d(90%) = 136.7 μm |
| Viscosity | Pseudo-plastic 603.7 | Pseudo-plastic 721 - 852 |
| | mPas (at 100 s⁻¹ | mPas (at 100 s⁻¹ room |
| | 90° C.) | temperature) |
| Molecular weight | | Mᵣ = 419,148 for |
| from intrinsic | | k = 0.0008 |
| viscosity [η] = kMᵣ^{α} | | α = 0.66 |

5-ASA: Mesalazine, also known as mesalamine or 5-aminosalicylic acid, and is an aminosalicylate anti-inflammatory drug used to treat inflammatory bowel disease, including ulcerative colitis, or inflamed anus or rectum, and to maintain remission in Crohn's disease.
Kollidon 30: Polyvinylpyrrolidone, with an average molecular weight expressed in terms of the K-value as in the pharmacopoeias valid in Europe, the USA and Japan, calculated from the relative viscosity in water in the range of 27.0-32.4.
Eudragit FS-30-D: is the aqueous dispersion of an anionic copolymer based on methyl acrylate, methyl methacrylate and methacrylic acid. The ratio of the free carboxyl groups to the ester groups is approx. 1:10. It is provided as an aqueous dispersion with 30% dry substance. The dispersion contains 0.3% Sodium Laurilsulfate Ph. Eur./NF and 1.2% Polysorbate 80 Ph. Eur./NF on solid substance, as emulsifiers. Based on SEC method the weight average molar mass (Mw) is approx. 280,000 g/mole.
Talc particle size: 99.5%<75 micrometer, median 19.3 micrometer, Ph. Eur. Specific surface (BET) 3.5 m2/g, producer: Imerys Talc, Italy SpA/Luzenac Pharma.

### Experimental Methods:

Drug release was measured in a USP2 apparatus at 37° C. with paddle rotation rate 100 rpm. One tablet per vessel was used. A four stage test was performed with the following medium composition:

In the first two hours the medium consisted of 900 mL 0.1 N HCl solution in purified water with pH 1.2.

In the following two hours the medium consisted of 900 mL 100 mM potassium phosphate monobasic adjusted to pH 6.5 with NaOH.

In the following two hours the pH of the medium was adjusted to 6.8 with NaOH.

In the last stage the medium was exchanged with 200 mL of the same phosphate buffer pH 6.8 that contained different concentrations of xyloglucanase. The latter was a microbial enzyme of Paenibacillus sp. that is specific for digestion of Xyloglucan. The unit titer is calibrated with tamarind xyloglucan.

The first test stage (pH 1.2) simulates the stomach environment while the second test stage (pH 6.5) simulates the passage through the upper small intestine. The pH 6.8 stage corresponds to the movement of the tablet to the lower small intestine and the last stage with the reduced fluid volume and the presence of microbial enzyme corresponds to the environment in the colon, where release shall take place.

### Results and Discussion:

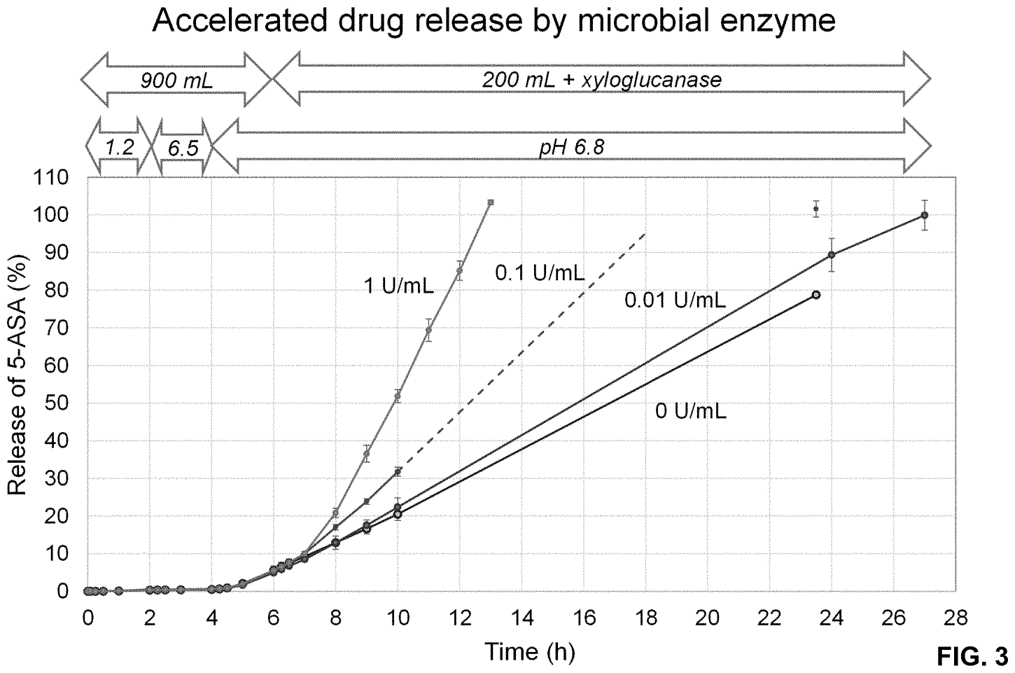

Typical results are shown in the graphic in FIG. 3. No drug is released in the conditions reflecting the stomach and the upper small intestine, this being due to the polymeric coating. The residence time of two hours in each of these environments is representative for the transit time of solid dosage forms in the gastrointestinal tract after a light meal as found by scintigraphy and other methods.

The coating film was designed to dissolve and be removed from the surface of the tablet at pH 6.8. This results in a modest release of API that did not exceed 10% in two hours. The rate of drug release was markedly accelerated in the presence of the microbial enzyme in a concentration dependent fashion providing the proof of principle of controlled and position-triggered drug release by the developed delivery system.

After dissolution of the polymeric coating, release is inhibited by the Xyloglucan that does not allow the tablet to disintegrate forming a highly viscous gel or gluey mass instead which acts as diffusion barrier. Although pH values as high as 7.2 have been reported for the distal small intestine the coating is deliberately designed to dissolve at lower pH, the reason being that pH values and residence times in the intestine may fluctuate and exhibit inter-individual variability, and that the pH in the ascending colon can be again <7. Hence, if a film coating does not dissolve in the small intestine in a timely fashion, the tablet may be defecated intact as observed already with other experimental systems.

For the present delivery system, an early, i.e. at low pH, removal of the coating does not result in excessive release of drug in the small intestine and thus an undesirable loss of API for local action in the colon as this is prevented by the Xyloglucan matrix. Upon entering the colon the action of localized microbiome-specific enzymes ensures a rapid drug release. Hence the synergistic overlap of two control mechanisms provides a highly targeted delivery to the colon. The employed enzyme concentrations correspond to those reported and reasonably expected to be found in the human large intestine.

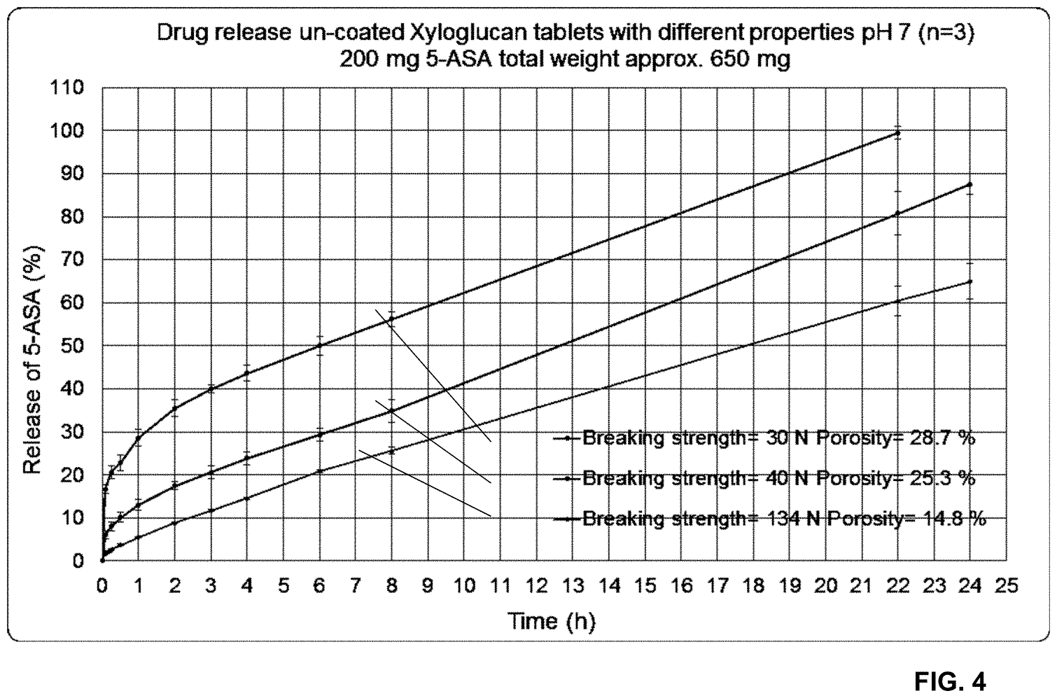

The property of Xyloglucan to slow down drug release is demonstrated in FIG. 4. Depending on the porosity of un-coated tablets release of the API can be adapted to take much longer than 24 hours while the release process follows approximately zero-order kinetics. The accelerating effect of enzymatic degradation of Xyloglucan on drug release is shown on FIG. 3.

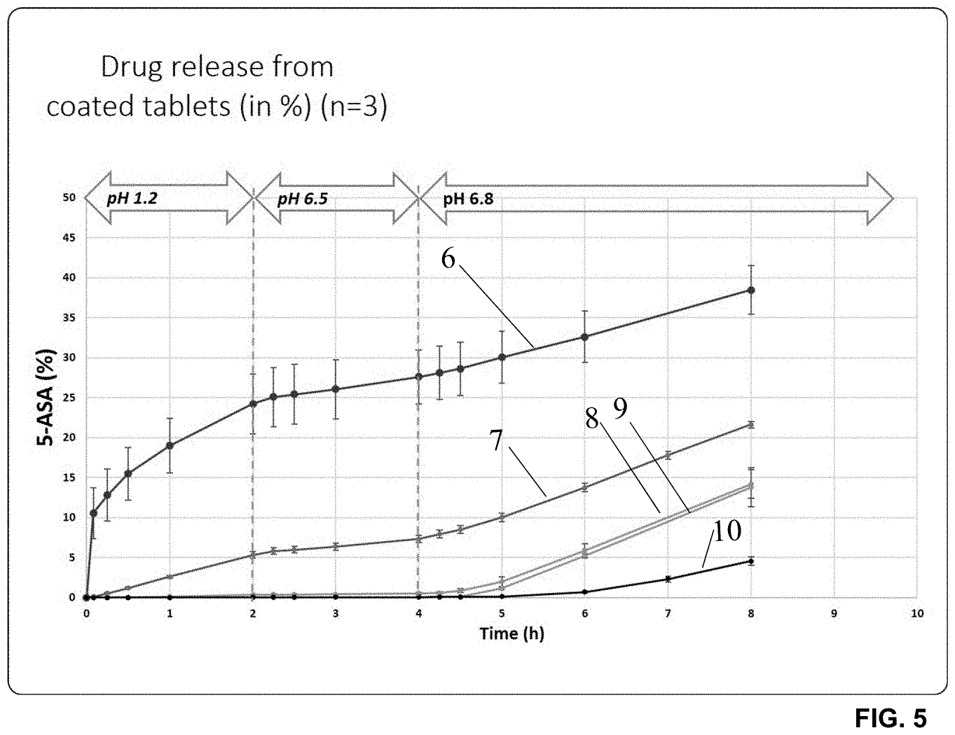

FIG. 5 shows the drug release from tables as a function of time and pH for various modifications, for no coating (6) and with coating (7-10) situations. All cores of the tablets had a crushing force of 50 N. The coating thickness defined as coating mass per square centimeter of tablet surface increased from 2 mg/cm² to 3.4. mg/cm² to 4.9 mg/cm² to 6.8 mg/cm² for curves 7 to 10, respectively. The results show the optimal coating thickness for effective enteric coating and timely dissolution of the coating. The results also show that no burst release and therefore better release control takes place after the coating is dissolved at pH 6.8 contrary to the absence of a coating at the same pH (pH 7), shown in FIG. 4, underlining the synergy effect between coating and xyloglucan.

Distinction from Prior Art:

As for the distinction from the above-mentioned Yoo publication the following is to be noted: The results of the Yoo publication prove that the manufactured product (beads) do not work like the tablets proposed here. In FIG. 5 of Yoo the release of the active substance is measured first in the simulated stomach medium pH 1.2 and then in the simulated intestinal medium pH 7.4. The coated beads of Yoo show a release of about 10% in the first 2 hours at pH 1.2 and about 50% in the next 2 hours at pH 7.4. The formulation as described here shows a release of 0% at pH 1.2 after 2 hours, a release of 0% at pH 6.5 after 2 hours and a release of about 5% at pH 6.8 after a further 2 hours. Since the goal is to have as little release as possible under intestinal conditions, the product in Yoo by far does not meet our requirements.

Furthermore the experiment from Yoo was replicated as follows using native xyloglucan as used here instead of the degalactosylated type of Yoo:
a 2% xyloglucan solution (quality 3S as in the tests here) in water was prepared at 4° C. while stirring with propeller stirrer during 24 hours.
plant oil was heated at 40° C. and at 80° C. under magnetic stirring.
1 mL of xyloglucan solution was dripped through a syringe with needle (ID 0.71 mm corresponding to 22G) to the oil.

The result has been documented photographically.

At both temperatures, drops with a size of about 2 to 2.5 mm are formed at the beginning.

At 40° C. the drops flow together to worms after a few minutes.

At 80° C. air bubbles have formed in the droplets, the droplets have risen to the surface and flowed together during filtering.

Based on this the following interpretation has to be drawn: Native xyloglucan (not de-galactosylated) does not gel. De-galactosylated xyloglucan forms gels. The temperature of gel formation depends on the degree of de galactolysis. This is confirmed by independent work (e.g. above mentioned Brun-Graeppi publication). The xyloglucan of Yoo has a 44% galactose removal and gels at 40° C. Native xyloglucan shows nothing up to 60° C. (FIG. 3 of Brun-Graeppi). We have gone up to 80° C. but also no gelation seen, gelation means a solidification of the drops. This prevents the droplets from flowing together. In Yoo the droplets are cured for 30 minutes at 40° C., filtered, washed with acetone and dehydrated with successive water/ethanol mixtures. This was not possible with the drops using the native xyloglucan because they flowed together and coalesced. The difference lies clearly in the de-galactosylation and the associated gelation, making the Yoo method impossible using native xyloglucan.

The small beads of Yoo are fundamentally different from the compressed tables given here at least for the following reasons: The beads from Yoo, not coated, with a drug load of 27.77% (Charge XGID 100, Table 1) show a drug release of above 70% in 2 hours at pH 7.4 (FIG. 2a). Our tablets with a drug load of 30% (comparable to the beads) show a drug release between 10 and 35% at pH 7 within 2 hours (also comparable to the beads, FIG. 4 given here) depending on the strength of the compression. Strongly compressed tablets with a high strength (134 N crushing force) have a porosity of 14.8% i.e. a relative density of 0.852 (space filling of 85.2%) and show a release in 2 hours of almost 10%. Slightly compressed tablets (crushing force=30 N, relative density=0.713) show a release in 2 hours of about 35%. The results clearly demonstrate the great influence of the volume and/or density of the tablets and the compression process on the release (without coating). The density of the beads must be significantly below 70% due to the manufacturing method (syringe method as given above). One starts with a 2% xyloglucan solution to which one adds a maximum of 2% active ingredient. With this, one cannot reach a solid content of 70 V-% in the beads at the end. This is visible from the very different amount of release between beads and tablets in general.

The release with coating also works differently with the beads of Yoo than with our tablets. FIG. 5 of Yoo shows release after 2 hours pH 1.2 (stomach conditions) with coating almost 10% and after further 2 hours at pH 7.4 (small intestine conditions) release of total 50%. In the same duration under the same conditions without coating the release from the beads is about 65%. This means firstly that the coating makes a relatively small difference (50 vs. 65%) and secondly that the goal of releasing as little active substance as possible in the small intestine is not achieved. In our tablets (FIG. 3 given here) we have a release of 0% at pH 1.2 after 2 hours, a release of 0% after 2 hours at pH 6.5 (upper conditions of the small intestine) and a release of about 5% after a further 2 hours at pH 6.8 (conditions of the small intestine). Without coating under the same conditions we have a release of about 32% (FIG. 5 given here). This means, firstly, that the coating makes up a great deal (5 vs. 32%) and, secondly, that we achieve the goal of the lowest possible release in the small intestine (during a total of 6 hours).

In addition, the presence of the coating influences the release after the coating is dissolved and removed, i.e. at pH 6.8. If one compares the release in FIG. 4 at pH 7 with the release at pH 6.8 in further FIG. 5, one can see that in the case of a coating the burst effect (sudden increase) is omitted at the beginning while afterwards the release rate in the case of the coating is somewhat higher (than without coating). The latter can also be seen in the presence of the microbial enzyme. This has to do with the fact that the inside of the tablet is still intact in the first 4 to 6 hours during the coating, absorbs water and the xyloglucan starts to swell which influences the subsequent release. So there is a synergistic effect between xyloglucan core and coating which influences the release.

In contrast, using the formulation given here, tablets (several mm diameter) of core and API are prepared and the core is later coated with an enteric film. The release of the API is as follows: No or only minor release is expected and observed at low pH (gastric passage).

Upon neutralization (entry in the small intestine) the enteric coating dissolves. Water will now wet the tablet and water will diffuse into the tablet. In the outer surface of the tablet core xyloglucan (solid, no air) will form a highly viscous mass impeding release of API and slowing down water intrusion. Consequently, there will be only a minor release of the API for several hours (“delayed release”).

The polysaccharide xyloglucan is not degraded by human digestive enzymes. Instead, xyloglucan and other plant cell wall derived polysaccharides are degraded and metabolized by the colonic microbiome. We could indeed show that in the presence of xyloglucanase (the enzyme which initiates degradation of xyloglucan) the API release is accelerated which is probably caused by the accelerated erosion of the xyloglucan matrix by the enzymatic degradation. The specific degradation of xyloglucan by the colonic microbiome constitutes the second control mechanism of our technology.

LIST OF REFERENCE SIGNS

| | |
|---|---|
| 1 | pharmaceutical formulation |
| | dosage form, tablet |
| 2 | core |
| 3 | shell |
| 4 | active pharmaceutical |
| | ingredient |
| 5 | matrix, xyloglucan |
| 6 | S-20-50N total mass 631 mg |
| | no coating |
| 7 | S-20-50N Eudragit F530, |
| | L = 2 Total mass 639 mg |
| | with coating |
| 8 | 5-20-50N Eudragit F530, |
| | L = 3.5 Total mass 651 mg |
| | with coating |
| 9 | S-19-50N Eudragit F530, |
| | L = 4.9 Total mass 671 mg |
| | with coating |
| 10 | S-20-50N Eudragit F530, |
| | L = 6.8 Total mass 661 mg |
| | with coating |

1. A pharmaceutical formulation dosage form with a core encapsulated by at least one shell and comprising at least one active pharmaceutical ingredient,
wherein the at least one active pharmaceutical ingredient is embedded in said core or forming said core of the pharmaceutical formulation dosage form,
wherein said shell comprises a pH-responsive coating,
and wherein at least one of said core and said shell is based on xyloglucan.

2. The pharmaceutical formulation dosage form according to claim 1, wherein the at least one active pharmaceutical ingredient is embedded in said core of the pharmaceutical formulation dosage form in that said core is formed by a matrix based on xyloglucan containing said active pharmaceutical ingredient.

3. The pharmaceutical formulation dosage form according to claim 1, wherein said shell comprises at least one outer layer in the form of a pH responsive coating, based on xyloglucan or free from xyloglucan, as well as at least one inner layer based on xyloglucan if the outer layer is free from xyloglucan.

4. The pharmaceutical formulation dosage form according to claim 1,
wherein it is adapted for oral administration and for targeted release of the active pharmaceutical ingredient in the colon, and
wherein said shell comprises at least one or consists of at least one pH-responsive coating dissolving only at a pH of more than 6.5.

5. The pharmaceutical formulation dosage form according to claim 1, wherein said shell is based on a synthetic polymer and/or a biopolymer or a mixture thereof.

6. The pharmaceutical formulation dosage form according to claim 1, wherein said shell consists of a mixture of an anionic acrylate copolymer with further additives in a proportion of less than 25%.

7. The pharmaceutical formulation dosage form according to claim 1, wherein the dry coating amount of the at least one pH responsive coating or of the whole shell (3) is in the range of 1-10 mg/cm2.

8. The pharmaceutical formulation dosage form according to claim 1, wherein there is provided only one single encapsulating pH responsive coating forming said shell.

9. The pharmaceutical formulation dosage form according to claim 1, wherein said matrix of the core essentially or completely consists of xyloglucan.

10. The pharmaceutical formulation dosage form according to claim 1, wherein the core consists of:
(A) 25-90% by weight of xyloglucan;
(B) 10-60% by weight of at least one active pharmaceutical ingredient; and
(C) 0-20% by weight of one or more pharmaceutically acceptable excipients;
or
wherein the core consists of granules consisting of:
(A) 25-90% by weight of xyloglucan;
(B) 10-60% by weight of at least one active pharmaceutical ingredient; and
(C) 0-20% by weight of one or more pharmaceutically acceptable excipients,
which granules are compacted to form a core before applying the shell.

11. The pharmaceutical formulation dosage form according to claim 1, wherein the weight ratio of the matrix of the core to the at least one active pharmaceutical ingredient is at least 1:2.

12. The pharmaceutical formulation dosage form according to claim 1 for the purpose of establishing, re-establishing and/or modifying the balance of the microbiome population in the colon or the physiology of the lower gastrointestinal tract, or for immunomodulation or immunosuppression or for the treatment of at least one of the following conditions: inflammatory bowel disease, in particular ulcerative colitis and/or Crohn's disease, Clostridium difficile infection, colon cancer, post colon surgical treatment.

13. The pharmaceutical formulation dosage form according to claim 1, wherein the active pharmaceutical ingredient is one or more selected from the group consisting of: mesalazine, budesonide, capecitabine, fluorouracil, irinotecan, oxaliplatin, UFT, cetuximab, panitumumab, immunomodulatory ingredients, immunosuppressive ingredients, immunosuppressive glucocorticoids, immunosuppressive cytostatics, immunosuppressive (poly- or monoclonal) antibodies, immunosuppressive drugs acting on immunophilins, interleukins, cytokines, chemokines, immunomodulatory imide drug, tacrolimus cyclosporin, materials for the purpose of establishing, reestablishing and/or modifying the balance of the microbiome population in the colon, and compounds which have a beneficial effect on the physiology of the lower gastrointestinal tract.

14. A method of treatment, comprising: administering the pharmaceutical formulation dosage form according to claim 1 to a patient in need thereof orally at least once a day, or twice a day, over a time span of at least one week, or at least two weeks, or at least two months or at least 1 year or even life-long.

15. A method for making a pharmaceutical formulation dosage form according to claim 1,
wherein in a first step xyloglucan, at least one active pharmaceutical ingredient, as well as if needed one or more pharmaceutically acceptable excipients are mixed and then compacted to form the core or mixed and treated to form granules, with an average diameter in the direction of the smallest diameter of at least 3 mm, which are subsequently, if needed by first mixing the granules with a further treatment agent, compacted to form the core,
and
wherein the core is subsequently coated in a second step with at least one coating forming a shell.

16. The pharmaceutical formulation dosage form according to claim 1, wherein the core is a single solid compressed core with a relative density of at least 0.7.

17. The pharmaceutical formulation dosage form according to claim 1, wherein the core or the whole pharmaceutical formulation dosage form has an average diameter in the direction of the smallest diameter of at least 3 mm.

18. The pharmaceutical formulation dosage form according to claim 1, wherein the core or the whole pharmaceutical formulation dosage form has a crushing force of at least 25N.

19. The pharmaceutical formulation dosage form according to claim 1, wherein the dosage form is adapted for oral administration and for targeted release of the active pharmaceutical ingredient in the colon, and wherein said shell comprises at least one or consists of a pH-responsive coating dissolving only at a pH of at least 6.7.

20. The pharmaceutical formulation dosage form according to claim 1, wherein the at least one pH responsive coating of the shell is based on a synthetic polymer and/or a biopolymer or a mixture thereof, based on an anionic acrylate copolymer.

21. The pharmaceutical formulation dosage form according to claim 20, wherein the anionic acrylate copolymer is based on methyl acrylate, methyl methacrylate and methacrylic acid, wherein the ratio of the free carboxyl groups to the ester groups is in the range of 1:5-1:10.

22. The pharmaceutical formulation dosage form according to claim 20, wherein the anionic acrylate copolymer has a weight average molar mass (Mw) in the range of 200,000-400,000 g/mole.

23. The pharmaceutical formulation dosage form according to claim 1, wherein the at least one pH responsive coating of the shell consists of a mixture of an anionic acrylate copolymer based on methyl acrylate, methyl methacrylate and methacrylic acid, wherein the ratio of the free carboxyl groups to the ester groups is in the range of 1:5-1:10, wherein the anionic acrylate copolymer has a weight average molar mass (Mw) in the range of 200,000-400,000 g/mole, with further additives in a proportion of less than 25%, said further additives being selected from the group consisting of polyoxyethylene and derivatives thereof, anionic surfactants, including sodium laurylsulfate, talc, dye, iron(III)oxide, stabilizers, and triethyl citrate.

24. The pharmaceutical formulation dosage form according to claim 1, wherein the dry coating amount of the at least one pH responsive coating or of the whole shell is in the range of 2.5-6 mg/cm2.

25. The pharmaceutical formulation dosage form according to claim 1, wherein said matrix of the core essentially or completely consists of xyloglucan, wherein said xyloglucan is obtained from Tamarindus indica seeds and/or is cold water soluble and/or is amorphous,
or wherein the xyloglucan used as starting material has a particle size (d50%) of at least 70 μm,
or wherein the xyloglucan has a weight average molar mass (Mw) in the range of 400,000-500,000 g/mol,
or wherein the xyloglucan is non-degalactosylated and/or native.

26. The pharmaceutical formulation dosage form according to claim 1, wherein said matrix of the core essentially or completely consists of xyloglucan which is a native, highly purified xyloglucan, of a cold-water soluble type.

27. The pharmaceutical formulation dosage form according to claim 1, wherein the core consists of:
(A) 40-90% by weight of xyloglucan;
(B) 10-60% by weight of at least one active pharmaceutical ingredient; and
(C) 5-10% by weight of one or more pharmaceutically acceptable excipients selected from the group consisting of a diluent, a binder, an anti-adherent, a lubricant, a glidant, and a combination thereof, including the situation where the pharmaceutically acceptable excipient essentially consists of a binder or a binder and an anti-adherent, wherein the binder can be selected as PVP and the anti-adherent as magnesium stearate, or
wherein the core consists of granules consisting of:
(A) 40-90% by weight of xyloglucan;
(B) 10-60% by weight of at least one active pharmaceutical ingredient; and
(C) 5-10% by weight of one or more pharmaceutically acceptable excipients selected from the group consisting of a diluent, a binder, a lubricant, a glidant and a combination thereof, including the situation where the pharmaceutically acceptable excipient essentially consists of a binder,
wherein the binder can be selected as PVP,
wherein the granules are compacted to form a core before applying the shell, and
wherein before compacting the granules can be blended with an anti-adherent, including in the form of magnesium stearate.

28. The pharmaceutical formulation dosage form according to claim 1, wherein the weight ratio of the matrix of the core to the at least one active pharmaceutical ingredient is at least 1:1.

29. A method for making a pharmaceutical formulation dosage form according to claim 1,
wherein in a first step xyloglucan, at least one active pharmaceutical ingredient, as well as one or more pharmaceutically acceptable excipients are mixed and then compacted to form the core or mixed and treated to form granules, compressed to a single core with a relative density of at least 0.7 and/or with an average diameter in the direction of the smallest diameter of at least 3 mm, which are subsequently, if needed by first mixing the granules with a further treatment agent, compacted to form the core, wherein the mixing in both cases can take place using a fluidised bed granulator or high shear mixer,
wherein the core is subsequently coated in a second step with at least one coating forming a shell, and
wherein the coating formulation can be provided as a dispersion and can be applied further in a drum coater.
